(19) United States
(12) Patent Application Publication
(10) Pub. No.: US 2023/0076642 A1
(43) Pub. Date: Mar. 9, 2023

(54) INTERACTIVE MULTI-PROBE ABLATION GUIDANCE SYSTEM AND METHOD

(71) Applicant: NE Scientific, LLC., Boston, MA (US)

(72) Inventor: Andrea Borsic, Torino (IT)

(73) Assignee: NE Scientific, LLC., Boston, MA (US)

(21) Appl. No.: 17/970,097

(22) Filed: Oct. 20, 2022

Related U.S. Application Data
(63) Continuation-in-part of application No. PCT/US2021/028452, filed on Apr. 21, 2021.
(60) Provisional application No. 63/013,863, filed on Apr. 22, 2020.

(51) Int. Cl. A61B 34/10; A61B 34/20

(52) U.S. Cl. CPC A61B 34/10; A61B 34/20; A61B 2018/00577

(57) ABSTRACT

The present invention is directed to a multi-probe ablation simulation and guidance system and method for use in tissue ablation procedures. In use, the relative locations of a plurality of ablation probes capable of providing ablation energy are determined, and the effect of energy provided by the probes based on the determined locations is predicted to identify a simulated ablation volume. This simulated ablation volume is compared with a target tissue volume. The relative locations of the probes can be adjusted based on the comparison between the simulated ablation volume and the target tissue volume, and the predicted effect rerun until the simulated ablation volume encompasses the target tissue volume to be ablated and necrotized.

### CROSS-REFERENCE TO RELATED APPLICATIONS

This application is a continuation-in-part of International Application No. PCT/US2021/028452, filed on Apr. 21, 2021, and entitled “INTERACTIVE MULTI-PROBE ABLATION GUIDANCE SYSTEM AND METHOD,” the entirety of which is incorporated herein by reference. International Application No. PCT/US2021/028452 claims the benefit of priority of U.S. Provisional Application 63/013,863, filed on Apr. 22, 2020, the entirety of which is incorporated herein by reference.

### FIELD OF THE INVENTION

The present invention is in the field of medicine. More particularly, the present invention generally relates to tissue ablation systems and therapeutic uses thereof.

### BACKGROUND

Existing tissue ablation technologies suffer from inaccuracy and inefficiency, which can result in procedures exceeding an hour in length, which is undesirable. This is so, at least in part, because of difficulties in predicting ablation volumes.

### SUMMARY OF THE DISCLOSURE

The present invention is directed to a multi-probe ablation simulation system and method for use to provide intraoperative guidance to clinicians during tissue ablation procedures. It has been discovered that treatment time and effectiveness can be improved by simulating ablation volume in real-time based on known probe positioning, by displaying the simulated ablation volume, and by continuing to update in real-time the simulated ablation volume to reflect any adjustments in the probe positioning. This allows a clinician to interactively adjust the probe positioning to ensure that a predicted and displayed treatment volume matches with the identified target volume.

This discovery has been exploited to develop the present disclosure, which, in part, is directed to a tissue ablation system and method for determining and delivering a therapeutic ablation volume, and for treating cancer, arrhythmia and other tissue diseases.

In one aspect of the present invention, the disclosure provides a tissue ablation system comprising: a multi-active probe controller; a plurality of ablation probes controlled by the controller; an imaging device; a screen for displaying computer generated information and/or medical images; a computing system for executing programming code and algorithms; and a facility picture, archiving and communication (PACS) network, with which the computing system interfaces.

In some embodiments, the ablation system is a microwave ablation system, a radiofrequency ablation system, a cryoablation), or an irreversible electroporation system. In some embodiments, the imaging device is a CT scanner, MRI scanner, a PET scanner, or ultrasound scanner device.

In certain embodiments, the screen is stand-alone component, part of a Personal Computer (PC), part of the ablation controller, or part of the imaging device.

In some embodiments, the computing system is a PC, an embedded system, a Virtual Machine, a Docker. In certain embodiments, the computing system is directly or virtually connected to the screen. In other embodiments, the computing system interfaces with the controller, or to at least one probe. In particular embodiments, the computing system interfaces locally or remotely to the facility PACS network. In some embodiments, the computing system interfaces locally or remotely to the imaging device.

In some embodiments, the ablation system further comprises a surgical tool tracking sub-system for tracking the intracorporeal position of the ablation probes.

In some embodiments, the ablation system uses an image processing method to identify the intracorporeal position of the ablation probes from images received from the imaging device.

In another aspect of the present invention, the disclosure provides a method for predicting the ablation volume of an ablation procedure. The method comprises determining relative locations of a plurality of ablation probes capable of providing ablation energy; predicting the effect of energy provided by the probes based on the determined locations to identify a simulated ablation volume; comparing the simulated ablation volume with a target tissue volume; adjusting the relative locations of said plurality of probes based on the comparison between the simulated ablation volume and the target tissue volume, and predicting an associated simulated ablation volume in connection with the adjusted locations until the simulated ablation volume encompasses the target tissue volume to be ablated and necrotized, where the simulation is conducted at an operational speed allowing for an interactive update of the simulated volume and its display reflect in real-time the positioning of the probes).

In yet another aspect of the present invention, the disclosure provides a method of ablating a tissue in a subject, wherein after the desired position of the plurality of probes is determined through simulation, the probes are placed in accordance with the determined positions and an ablation treatment operation is carried out. The method comprises identifying the position of at least two multi-active probes, the probes having a known geometry; simulating the ablation process in the tissue with a computing device, the computing device using the electrical and thermal characteristics of the tissue and the acquired probe positions, the probe geometries, the electrical and thermal characteristics of the probes, and the ablation power to be applied to each probe to provide an ablation a prediction of the corresponding tissue that would be necrotized if the ablation were performed; obtaining a visual display of the ablation volume; adjusting the position of, and/or energy provided by, the probes if the displayed simulation of necrotized tissue does not encompass the tissue to be ablated; repeating the simulating and obtaining a visual display steps until the projected ablation volume encompasses the target tissues, or anyways is judged adequate by the clinician; and performing the ablation.

In some embodiments, the ablation system is a microwave ablation system, a radiofrequency ablation system, a cryoablation, or an irreversible electroporation system.

In some embodiments, the probes have a known geometry, and in other embodiments, the probe geometry is determined from the image. In certain embodiments, the positions of the probes are identified with a surgical tool tracking system. In some embodiments, the positions of the probes are identified by acquiring an image of the tissue comprising the probes, and retrieving and processing the image with the computing device. In some embodiments, the computing devices uses the electrical and thermal characteristics of the tissue and the acquired probe positions, the probe geometries, the electrical and thermal characteristics of the probes, and the ablation power to be applied to each probe to provide the ablation prediction. In particular embodiments, the visual display is displayed by a screen or monitor, and in specific embodiments, the visual display is a 2D or 3D display. In some embodiments, the target tissue is a solid cancer or a tumor, and in other embodiments, the target tissue is the atrium of the heart.

In some embodiments, the method further comprises obtaining an image of the tissue; superimposing to this image a visual representation of the ablation volume, and displaying the so formed image. In certain embodiments, the method further comprising conducting additional simulating and displaying steps simultaneously with the ablation step.

In yet other aspects, the disclosure provides a method of treating a solid cancer in a subject using the tissue ablation system as described above. In still other aspects, the disclosure provides a method of treating arrhythmia in a subject using the tissue ablation system according to the disclosure.

These and other features of the present invention are described with reference to the drawings of embodiments of a multi-probe ablation simulation system. The illustrated embodiments of features of the present invention are intended to illustrate, but not limit the invention.

### BRIEF DESCRIPTION OF THE DRAWINGS

The foregoing and other objects of the present disclosure, the various features thereof, as well as the disclosure itself may be more fully understood from the following description, when read together with the accompanying drawings in which:

FIG. 1 is a diagrammatic representation of a representative prior art ablation volume indication resulting from the deployment of three multi-active probes as provided by the manufacturer of a commercially available ablation system, showing an axial plane orthogonal to the shaft the ablation probes, where the shafts intersect the plane and are represented as a dark dots and the ablation volume is indicated in the plane shown;

FIGS. 2A-2D illustrate the simulated positioning of two probes in a tissue, as instructed by the manufacturers of commercially available ablation systems, where the probes are required to be parallel to each other and/or to be positioned such that the distal ends are aligned in a common plane, with FIGS. 2A and 2C showing the probes in proper relative positions and FIGS. 2B and 2D showing the probes in improper relative positions;

FIG. 3 is a diagrammatic representation of the architecture of the present system;

FIG. 4 is a diagrammatic representation of the flow of information through the system as the simulation proceeds;

FIG. 5 is a diagrammatic representation for the alternative methods provided by the disclosure for estimating the position of the probes;

FIG. 6 is a representation of the detection result of a probe, wherein the two views shown are generated by slicing the 3D CT image volume in two orthogonal planes passing by the shaft of the probe (visible as a high intensity feature in the image), wherein the detected position of the shaft of the probe is indicated with a red line, the and the correct recognition of the probe position is confirmed by the concordant alignment of the probe in the CT images and of the red line generated by the guidance software;

FIG. 7 is a diagrammatic representation of the steps involved in the computation of the ablation volume;

FIG. 8 is a simulation provided by the present method showing a model of the ablation probes translated to the detected position of some real probes deployed in a liver, where the liver under ablation is simulated with FEM mesh generated from images of the organ in a patient;

FIGS. 9A-9B are representations of generated computer models showing the simulated positioning of three probes deployed in liver tissue, where the probes are not parallel, and their spatial position does not conform to any particular indication;

FIGS. 10A-10B are representations of generated computer models showing the simulated ablation results for the three probes, as positioned in FIGS. 9A and 9B; and indicating the ablation volume and thus which portion of the tissue is necrotized when the probes are activated, where the ablated (necrotized) tissue is indicated in pink, while the healthy liver is indicated in brown;

FIG. 11 is a representation of a computer model of a 2D CT image with a simulated overlay simulating what liver tissues would be necrotized (highlighted in yellow) as the results of an ablation with the positioned ablation probes; and

FIG. 12 is a block diagram of a computing system that can be used to implement any one or more of the methodologies disclosed herein and any one or more portions thereof. The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Tissue ablation technologies, such as radiofrequency ablation (RFA), microwave ablation (MWA), cryoablation (CRA), and irreversible electroporation (IRE), are used for necrotization of tissue for medical treatment purposes. For example, in the context of cancer treatment, these ablation technologies are used to kill a tumor by necrotizing the tumor, itself, and possibly some tissue margins around the tumor. In the context of cardiac arrhythmia, ablation technologies are similarly used to necrotize certain tissues of the heart that are responsible for disrupting a regular heartbeat.

In most tissue ablation technologies, energy is applied to tissues by the use of an ablation probe, such as a needle probe. When using such a probe, the clinician positions or “deploys” the probe, for example, percutaneously, and then activates it to apply energy. As ablative energy is applied to the probe, the surrounding tissue is necrotized.

In oncological contexts, the target tissue volumes can be relatively large as a tumor can have the diameter of several centimeters. In order to fully necrotize tissues having such larger volumes, clinicians commonly perform multiple overlapping ablations such that the resulting ablation volume encompasses the entire target tissues. This often requires positioning and activating the probe multiple times in order to cover the entire target volume.

The mechanisms by which tissues are necrotized by different ablation technologies are variable, but, for any one ablation technology, typically the clinician needs to apply energy for about 5 to 10 minutes for each ablation session in order for the ablation to be complete. Therefore, the total time for treating a larger target volume tissue increases in proportion to the number of overlapping ablations needed to treat that volume, and can exceed one hour, which is undesirable.

As a result of the time that is often needed for larger target tissue volumes, methods have been developed using multiple ablation probes that can be simultaneously activated (“multi-active probes”). When using these multi-probe systems, the clinician positions or “deploys” the multiple probes, again, for example, percutaneously, and then activates them at the same time, generally from a common controller. As ablative energy is applied to all the probes at the same time, the overall ablation time is reduced significantly, as the multiple ablations are not conducted in a serial fashion but in a parallel, collective fashion. Such systems reduce the overall ablation time needed to treat larger volume tissues when multiple ablations are required.

Clinicians aim to deploy the probes in such a way that the ablation volume created by deploying each probe will overlap the ablation volumes produced by the other deployed probes. This overlap is necessary to produce a necrotization volume that has no gaps of untreated tissues. As the multi-active probes are deployed in the same relative vicinity (to cause an overlap of ablation volumes), there is some interaction between the effects of each probe, and, as a result, the resulting ablation volume is not simply the sum of all the ablation volumes of each probe if each were operated independently. Rather, the overall ablation volume depends, in part, on the relative positions of each multi-active probe involved in the ablation.

In multi-active probe systems, the number of probes can range, for example, from two to 20, with a typical multi-active probe system having two to four probes. The manufacturer of such systems typically provides information about how to attain a representative ablation volume given the number of probes, and requiring that that the probes are in certain orientations, which are used by clinicians to plan and conduct the ablation. These constraints all together result in a given and unique spatial configuration of the probes which correlates to specific data for which the manufacturer indicates the resulting ablation volume. However, in practice, even if the representative ablation volume provided by the manufacturer were desired, which it may not, it is difficult and time-consuming for the clinician to place and orient the probes as instructed to meet these requirements. This is because vessels, bones, and other body structures affect the possible positioning of the probes, and it might not possible or desirable for the clinician to place the probes as required by the manufacturer. For example, when using three probes, the probes generally need to be placed in a perfect equilateral triangle. However, arranging the probes in such perfect relationship is difficult in actual practice. Thus, the true ablation volume will depart from the indicated ablation volume.

In addition, as the ablation volume is three-dimensional and has a complex shape, especially when using multiple probes at the same time, manufacturers typically offer an indication of a single cross-section, for simplicity. However, ablation volume will vary across sections more distal or proximal along the shaft of the probes. As a result, this inconsistency may result in untreated or overtreated tissue.

Further, in an effort to match the manufacturer's prescribed deployment geometry, the clinician may extract and insert one or multiple probes multiple times to get a deployment closer to what is indicated by the manufacturer. The act of inserting a probe into a tissue such as a tumor has inherent risks, by itself, including bleeding of the punctured tissue and track seeding of malignant cells from the tumor to distal body locations. Having to execute multiple deployments of the probes only increases the risk and incidence of these problems. Additionally, as noted above, multiple deployments add time onto the treatment. In current practice, a clinician places the probes in the patient and builds a mental map of which tissues would be ablated from printed information indicating the ablation volume provided by the ablation system/probe manufacturer. This approach is clearly unsatisfactory, as the mapping of information from paper to the three-dimensional nature of the target tissues is clearly not an easy process to be conducted mentally, and such an approach leads to imprecision. Thus, what is needed are systems and methods for more accurately predicting and indicating in real-time directly over the patient images the ablation volume of an ablation procedure, as well as improved methods of treating cancer and other disorders using these systems and methods. Further, there is a need for simulation of the ablative effects of a multi-probe deployment relative to a target tissue volume that can be run and adjusted, as needed, prior to treatment. Still further, the is a need for such a system, and associated methods, that will assist the clinician in identifying and executing a deployment of multiple ablation probes relative to a target tissue volume.

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

Tissue Ablation Systems

Ablation systems deliver energy and obtain the necrotization of tissues by heating (radiofrequency, microwave ablation), by freezing (cryoablation), and by causing irreversible cell damage (electroporation ablation) through one or more probes which necrotize a certain volume of targeted tissue. Prediction of the volume that could be necrotized by the ablation procedure is representative of the effect of the probes, and can be used to properly treat the target tissues and to lessen the volume of non-target tissue affected by the procedure. Predictions of the necrotized volume assist the clinician in proper placement of the probes prior to treatment, with the goal of maximizing the volume of treated target tissue, minimizing the volume of treated non-target tissues, and of reducing the overall treatment time. A non-optimal placement of the probes could require additional treatment steps in order to cover the entire treatment tissue volume. Similarly, using the non-optimal placement of the probes could also treat larger volumes of non-target tissue, or fail to treat the target tissues.

Prediction of ablation volume and/or any other processes and/or system elements may be implemented, without limitation, as disclosed in International Application No. PCT/US21/63257, filed on Dec. 14, 2021, and entitled “SYSTEM AND METHOD FOR ABLATION TREATMENT OF TISSUE WITH INTERACTIVE GUIDANCE,” the entirety of which is incorporated herein by reference.

The manufacturers of existing ablation systems provide predictive information graphically depicting the ablation volume that can potentially be delivered by a certain number of strategically and specifically positioned probes at certain operation parameters. Typically, the manufacturer provides information for one representative ablation volume for a single and given positioning of the probes of the system. However, a desired ablation volume (matching a three-dimensional target tissue volume) is likely different than the one provided by the manufacturer. This specific desired volume will depend on the relative position of all the probes involved, and there are infinite numbers of possible relative positions in which the probes can be deployed.

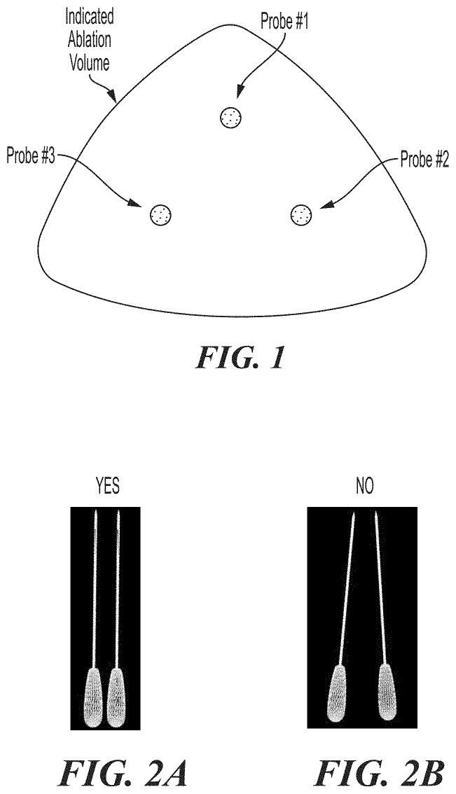

For example, it is common to use three probes for an ablation procedure. In accordance with manufacturer suggested usage of three probes, the probes should ideally be placed in an equilateral triangle, as illustrated in FIG. 1. For systems with four probes, it is common to indicate an ablation volume resulting from the probes being placed to form a square. The indication in FIG. 1 shows an axial plane orthogonal to the shafts of the ablation probes (i.e., the shafts intersect the plane and are represented in FIG. 1 as dark dots). The resulting ablation volume generated by normal simultaneous operation of the probes is indicated in the plane considered. In reality, the ablation volume is three-dimensional, but with many existing systems, only the two-dimensional representation illustrated in FIG. 1 is available.

In operation, the clinician is required to place the probes in spatial positions which are as close as possible to the one depicted in the figure (in this case, so the probe shafts form a triangle) in order to obtain the ablation volume as indicated. Positioning the probes at spatial locations which are different from those depicted in the figure results in a different and unknown ablation volume.

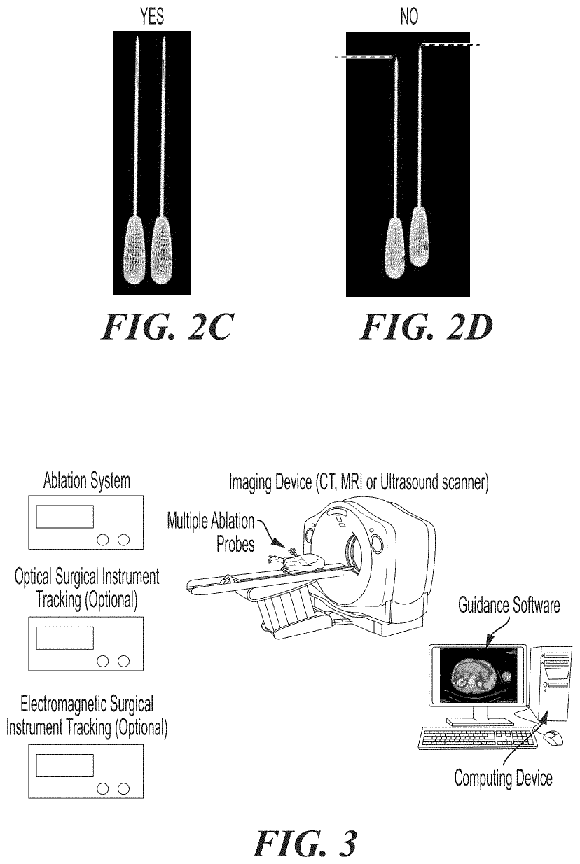

Besides positioning of the probes, the manufacturer further typically requires that the probes are parallel to each other and that the distal ends of the probes are aligned in the same plane. Proper probe positioning is indicated in FIGS. 2A and 2C. Improper positioning of the probes is indicated in FIGS. 2B and 2D. These constraints all together result in a given and unique spatial configuration of the probes, which is the specific one for which the manufacturer indicates the resulting predictive ablation volume used by a clinician in determining if the probe placement is properly aligned with a target tissue volume or if the probes need to be repositioned. Again, each time the probes require repositioning adds time to the treatment procedure.

However, in practice, even if the representative ablation volume provided by the manufacturer were desired, which it may not, it is difficult and time-consuming for the clinician to place the probes as instructed to meet these requirements. This is because vessels, bones, and other body structures affect the possible positioning of the probes, and it might not be possible or desirable for the clinician to place the probes as required by the manufacturer. Thus, the actual true ablation volume will typically depart from the indicated ablation volume. In addition, as the ablation volume is three-dimensional and has a complex shape, for simplicity, manufacturers offer an indication of a single cross-section, as in FIG. 1. Thus, the ablation volume will vary in other cross sections more distal or proximal along the shaft of the probes. As a result, targeted tissue may be left untreated, requiring additional treatment steps, or is overtreated, resulting in damage on normal tissue.

The present disclosure provides a remedy to such ablation volume uncertainty and its potential resulting damage to normal tissues or inadequate treatment of targeted ones. The disclosure provides a tissue ablation system for determining an accurate ablation volume given the positioning of multiple ablation probes.

In accordance with the disclosure, the system uses models and computer simulations to compute the ablation volume resulting from any actual positioning of the probes, and to indicate to the clinician running the system the computed volume on a screen. This “on-the-fly” approach does not require the clinician to match the probe configuration provided by the manufacturer (which is likely not the most accurate for necrotizing the present ablation target). Instead, this system enables the clinician to predict an ablation volume resulting from any one of an infinite number of probe positions that multiple probes can assume with respect to each other. In this regard, the clinician can simulate probe positioning and adjustment, and predict associated ablation and treated three-dimensional volumes, without needing to physically position and reposition the probes. More particularly, when the clinician has deployed the probes, the system indicates on a screen the exact ablation volume computed for the particular configuration of the probes. As the ablation volume is computed using a model of the physics, it is available as a three-dimensional (3D) shape on the system, and is displayed in 3D or in any 2D section as needed, without the uncertainty of how tissues outside of the cross-plane showed in representative graphical information provided by the manufacturer will be affected by the ablation. As a result, the clinician can adjust the position of the probes in the simulation to see how the computed ablation volume changes relative to the target tissue volume and determine an optimal placement for the probes. The simulation is conducted at intended operational speed, where the displayed simulated volume updates in real-time as the clinician adjust the simulated positioning of the probes, allowing an interactive assessment of the effects. Still further, the system can also assist the clinician execute the desired placement of the probes based on the simulation.

In order to compute the ablation volume, the computer models use the spatial positioning of the multi-active probes to be used in the ablation procedure. This information is available, for example, by processing intraoperative images which capture the ablation probes (for example, in CT images probes appear as high contrast objects and their shaft is identifiable because if its tubular shape and elevated contrast; alternatively Deep Learning networks can be easily trained to recognize surgical instruments in images), or by using common surgical tool-tracking technologies such as, but not limited to, the Stealth Station (Medtronic, 710 Medtronic Parkway, Minneapolis, Minn., USA), or the Aurora system (NDI Medical, 103 Randall Drive, Waterloo, Ontario, Canada, N2V 1C5) based on optical or electromagnetic methods.

The system of the present disclosure provides a tissue ablation system comprising: a multi-active probe controller; a plurality of ablation probes controlled by the controller; an imaging device; a screen for displaying computer generated information and/or medical images; a computing system for executing programming code and algorithms; and a facility PAC network, to which the computing system interfaces.

Controller

The ablation system is based on the type of energy it deploys and whether it could be effective to necrotize the volume of tissue targeted. As noted, typical ablation techniques include radiofrequency ablation (RFA), microwave ablation (MWA), cryoblation (CRA), and irreversible electroporation (IRE). Some representative ablation systems include microwave ablation systems like the Solero (AngioDynamics, 14 Plaza Drive, Latham, N.Y., 12110, USA), a radiofrequency ablation system like the Accurian (Medtronic, 710 Medtronic Parkway Minneapolis, Minn. 55432, USA), a cryoablation system like the Visual ICE (Boston Scientific, 300 Boston Scientific Way, Marlborough, Mass. 01752, USA), or an irreversible electroporation system like the NanoKnife (AngioDynamics, 14 Plaza Drive, Latham, N.Y. 12110, USA). In the example of an RFA system, the controller supplied RF energy to one or more probes deployed into the patient, e.g., Accurian probes from Medtronic for RFA, or e.g., NeuWave probes from Johnson & Johnson for MWA, or e.g., Visual ICE probes from Boston Scientific for CRA, or e.g., NanoKnife probes from AngioDynamics for IRE. The clinician can adjust the energy levels, but generally procedures are conducted using manufacturer set levels.

Probes

Ablation procedures are generally run with one to 20 probes. Most common procedures used between two and four probes. In such techniques, probes are multi-active and are selected for their size and geometry of their ablation footprint, which can range in diameter typically from 1 mm to 50 mm and in length from 1 mm to 70 mm. The number of probes used generally is based on the volume of tissue being targeted and which would most efficiently and effectively deploy the energy needed to necrotize the target tissue, given the known electrical and thermal characteristics of the tissue, as well as the in situ physics of the target tissue.

In accordance with the present invention, simulating the ablation treatment volume prior to actual treatment requires a determination of the specific positioning of the probes in the patient. Accordingly, the positioning of the probes in the tissue and their orientation relative to each other are determined using known techniques. For example, the position of the probes can be identified with an optical or electromagnetic surgical tool tracking system. Alternatively, the probe positioning can be identified by acquiring an image of the tissue comprising the probes, and retrieving and processing the image with a computing device. For example, in accordance with known techniques, probes can be placed in a patient, and the positions of the probes can be verified using a CT scan provided on the clinician's workstation.

Once the position of the probes is determined, the level of ablation power to be applied to each probe and the temporal modulation or each probe can be simulated volume.

Imaging Device

In various examples provided herein, the imaging device is a CT, Mill, PET, or ultrasound scanner device which is used by the clinician to acquire images of the patient during the procedure. The images acquired by the imaging device can also capture ablation probes deployed in the body, and these images can be used to determine the spatial position of the probes.

Screen

In accordance with the present invention, the ablation simulation system provides a simulation of ablation treatment in comparison with a target tissue volume. In this regard, the clinician places ablation probes in the patient and determines the relative positioning of the probes using an imaging device, such as a CT, MM, PET or ultrasound scanner device. An image showing the positioning of the probes is provided on a screen. In embodiments of the present invention, the screen is stand-alone component, part of the Personal Computer (PC), part of the ablation controller, or part of the imaging device. As noted, the image identifies the position of the probes in or relative to the target tissue, and further in accordance with the present invention, a simulated ablation volume, created by predicting ablative operation using the probes as so positioned, can be overlaid onto the image on the screen. Still further, the clinician can adjust the positioning of the probes, either in real time or via simulation using the computing system. If in real time, the clinician need sot remove and reposition the probes, and then take a new image using the imaging device. Then, the simulated ablation is re-run based on the new probe positioning. If via simulation, the positioning of the probes can be virtually adjusted and the simulation re-run until a desired probe positioning, with the predicted ablation volume overlapping the target tissue volume, is identified.

Computing System

The ablation simulation system in accordance with the present invention utilizes a computing system to predict ablation effects for probes positioned in a patient and overlaying such a predicted ablation volume over a target tissue volume so that a clinician can determine if probe positions are optimal or need further adjustment. The predicted ablation volume utilizes manufacturer data associated with the probes in connection with known operation of such probes. That is, the computing system is loaded with appropriate data so that intended operation of ablation probes can be simulated to show a predicted ablation volume with said probes operating at preprogrammed specifications.

The Computing System can interface to the Ablation Controller in order to receive data characterizing the ablation process, like for example the applied power, the duration of the ablation process, temperature of the tissues, electrical impedance seen at a probe, or electromagnetic reflection coefficient seen at a probe.

The Computing System may require the clinical to enter in a Graphical User Interface certain parameters describing the ablation process, such as, but not limited to, the specific probe in use, the amount of ablation power applied, and the duration of the ablation.

In examples of the present disclosure, the computing system is a PC, an embedded system, a Virtual Machine, a Docker. For example, the computing system is directly or virtually connected to the screen. In other embodiments, the computing system interfaces with the controller, or to at least one probe. The computing system can interface locally or remotely to the facility PACS network and/or to the imaging device.

In order to enable the clinician's assessment of the effects of different positioning of the probes, the Computing System and the algorithms predicting the ablation volume is capable of performing simulations in real-time, such that the displayed ablation volume can be updated at interactive rates when the simulated or actual position of the probes is updated.

Facility PACS Network

The facility PACS Picture Archival and Communication System (PACS) is a system which includes a network allowing the transmission, storage, and retrieval of medical images in common formats, like the DICOM (Digital Imaging and Communications in Medicine) format. The PACS network allows the computing device to receive medical images either directly from the imaging device, or to retrieve images stored on PACS storage nodes.

Surgical Tool-Tracking Subsystem

A surgical tool-tracking system continuously tracks the spatial position of the surgical tools. Optical systems are based on attaching to the part of the instrument that remains outside of the body of the patient special fiducials, which are recognized by multiple cameras, allowing a precise estimation of the position/orientation in space of such fiducials, and therefore of the tool. Electromagnetic tracking is instead based on setting up an array of electromagnetic coils, for example on the surface of the operating table, and in equipping the surgical tools with multiple miniaturized receiving coils. Analysis of the received signals at the coils allows to determine with precision the position/orientation of the surgical tool. In the present system, a surgical tool-tracking system continuously tracks the spatial position of the ablation probes and communicate this position via network to the computing device/guidance software. One useful tracking system is StealthStation (Medtronic, 710 Medtronic Parkway, Minneapolis, Minn., USA). Optionally an electromagnetic surgical tracking system like the Aurora (NDI Medical, 103 Randall Drive, Waterloo, Ontario, Canada, N2V 1C5) can be used. This system continuously tracks the position of the ablation probes and communicates this position via network to the computing device/guidance software.

Ablation Volume Simulation and Tissue Ablation Methods

The disclosure also provides methods of simulating an ablation volume and of ablating a target tissue in a patient in need thereof.

Target tissues include any tissue, the ablation of which may be therapeutic. For example, solid cancers and tumors on or in any organ that can be treated by necrotizing them using the present system. The target tissue can be all or part of a tissue area such as a solid tumor or heart. The target tissue can also include a tissue outside of the original tissue, such as an area around or in contact with the tumor or atrium of the heart. In addition, atrial and supraventricular arrhythmias can be treated by ablating a portion of the atrial tissue of the heart. Ablation of endometrial tissue can be used to treat endometriosis.

The present methods comprise using a computed simulation to determine if the volume of tissue that is targeted by the particular ablation system being used will, in fact, be sufficient to necrotize the targeted tissue. The simulation identifies the relative placement of ablation probes in the tissue, and then computes, using manufacturer operation data, the necrotized volume for ablation.

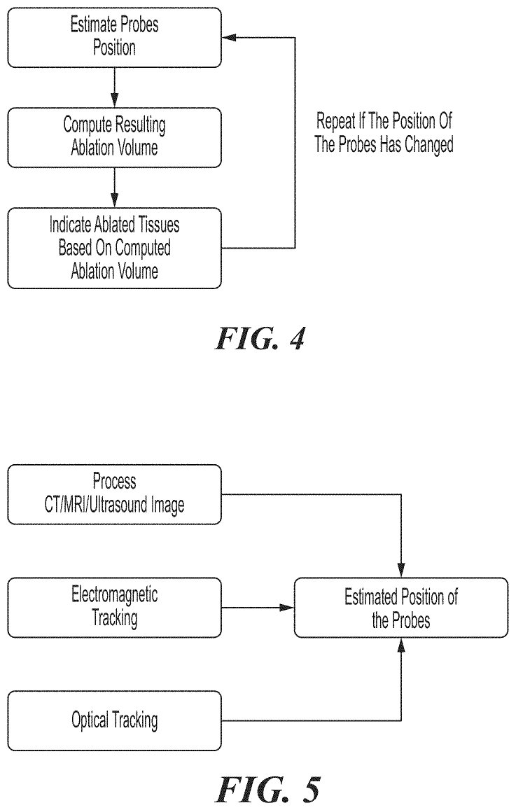

The general information flow of the system can be described in three steps, as illustrated in FIG. 4. In a first step the position of the ablation probes is estimated. The ablation volume resulting from the position of the ablation probes is then computed. The ablated tissues are indicated (for example by highlighting them on the images of the patient, based on the computed ablation volume, and other parameters that affect the ablation volume).

The steps can be used in a planning setting, where the clinician might be adjusting the position of the probes; in this case the steps are repeated so that the indication of the ablated tissues reflects the current position of the probes.

Once the probes are adjusted in a chosen position, the clinician will stop adjusting their position and activate them. After the ablation is complete, or while it is ongoing, the steps are optionally performed one or more times in order to update the computed ablation volume with respect to physiological changes in the tissues or body, or with respect to variable parameters in the ablation system, changes that can occur during or as a result of the ablation. The position of the probes is assumed to be fixed during the ablation.

Estimating the Position of the Probes

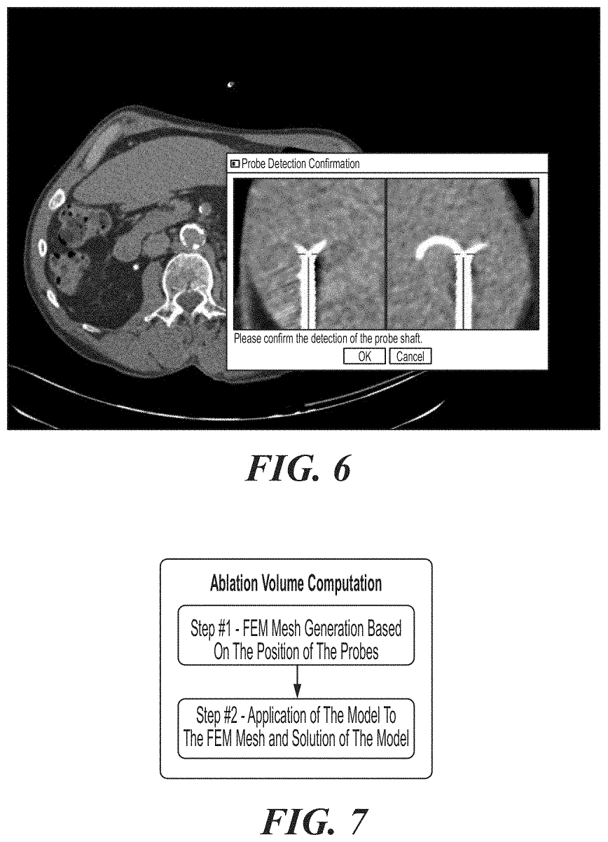

The present system provides three alternative ways of estimating the position of the probes, as shown in FIG. 5. A first method uses intraoperative images which capture the probes as deployed in the tissues. By processing these images on the computing device, algorithms detect the probes in the image and estimate their intracorporeal position. This can be achieved with traditional algorithms, or with Artificial Intelligence (AI)/Deep Learning (DL) algorithms. FIG. 6 shows a dialog in the system which is displayed to the user in order to confirm the detection of the position of the ablation probe (in the CT image is shown a LeVeen radiofrequency ablation probe from Boston Scientific, 300 Boston Scientific Way, Marlborough, Mass. 01752, USA).

An alternative method relies on off-the-shelf optical instrument surgical tracking technologies, where the tracking system would continuously stream the estimated position of the probes to the computing device via network protocols.

Another alternative relies on off-the-shelf optical instrument surgical tracking technologies, where the tracking system would continuously stream the estimated position of the probes to the computing device via network protocols.

For planning purposes, the adjustment of the relative locations of the plurality of probes can be theoretical in accordance with the present invention, in that the clinician need not physically move the probes for each adjustment. Instead, the relative positioning of the probes can be adjusted on the system itself, for example on the computer screen on which the target tissue is displayed, with the adjusted probe positions and the associated simulated ablation volumes for such positions overlaid onto the image of the target tissue volume.

Computing the Ablation Volume

The ablation volume is computed using models that reflect the physics of the ablation. The system supports Radio Frequency Ablation (RFA), Microwave Ablation (MWA), Cryo-Ablation (CRA), and Irreversible Electro Poration (IRE). The models, which are described by Partial Differential Equations (PDEs) and by their boundary conditions, are discretized in our implementation (with no loss of generality) using the Finite Element Method (FEM) in the spatial domain, and using the Finite Difference Method in the temporal domain.

The computation of the ablation volume consists in two steps: in the generation of an FEM mesh that models the probes in their position, and the tissues involved in the ablation, and in a second step which is the application of the model to the FEM mesh and its solution. This process illustrated in FIG. 7.

Generation of the Mesh

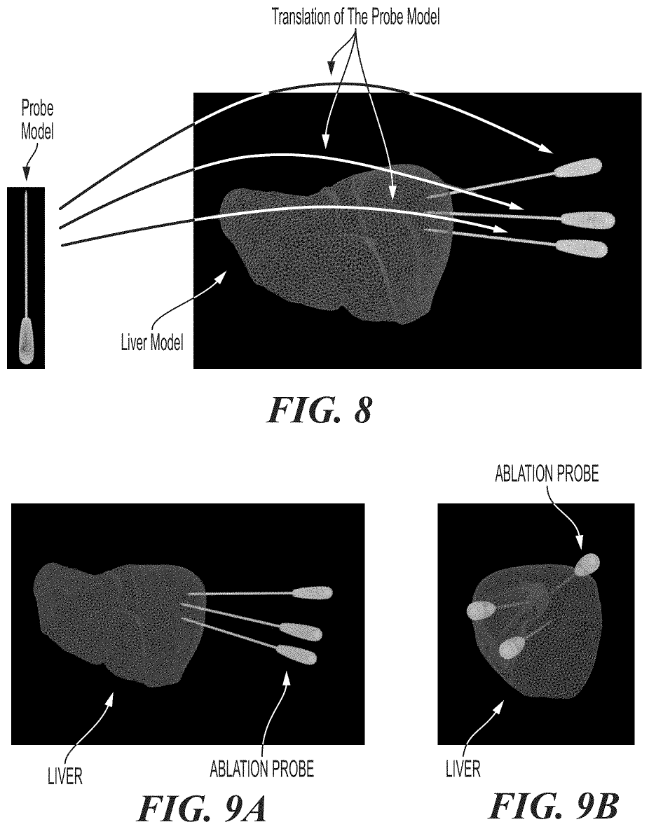

The FEM mesh captures the geometry and properties of the ablation probe in use and the properties of the tissues. The tissues can be modeled in a neighborhood of the of the ablation site, for example in a spherical domain centered at the ablation site. Alternatively, the organ under ablation can be modeled in full (FIGS. 8 and 9) with or without adjacent organs.

Pre-prepared models of the ablation probes obtained from different vendors, including the geometry and electrical and thermal properties of the materials from which the probe is made, are used in the system. These models are generated in a standard spatial position, which in the system, corresponds to a probe orientation along the z axis, and a position of the tip at the point (0,0,0). Based on the position of each probe deployed in the body, the probe model is translated to match that position, so that it represents the real position of the ablation probe as deployed in the tissues. The model of the tissues and the translated models of the probes are then used to generate a volume FEM mesh which is used for computational purposes in applying the model to the FEM mesh. As shown in FIG. 8, the organ being ablated (the liver in this case) is simulated with a FEM mesh generated from images of the patient. Without loss off generality only a portion of the organ might be simulated, or in addition to the organ, other adjacent organs could be modeled as well, particularly when the ablation might take place in proximity of the boundary between two organs.

Application of the Model to the FEM Mesh and Solution of the Model

Once a FEM mesh capturing the probes in their positions is created, it is used for computing the ablation volume resulting from those probe positions. Depending on the ablation physics being simulated (RFA, MWA, CRA, IRE) a different model representing those physics is applied (EXAMPLE 2).

Each of these models, when applied to the FEM mesh, result in a scalar field over the volume of the mesh which describes the tissue damage. This information can be used to display the damage, and can be combined with, or overlaid on, additional images of the organ, such as, but not limited to, CT images.

Thus, in accordance with the present disclosure, the relative placement of the probes is determined from a CT scan or similar image taken of the patient with probes in place. The simulated ablation volume is provided on a screen for displaying computer generated information and/or medical images, for example, a PC or workstation proximate to the patient. The simulated ablation volume is projected onto the CT scan, or a similar image, so that the clinician can see the target tissue volume, the current placement of the probes, and the simulated ablation volume overlaid on the same image. This projection helps the clinician determine if all the target tissue will be affected by an ablation treatment with the probes as so position. If the ablation volume does not match the target tissue volume, or anyways the desired treatment volume, the clinician can identify where the volumes are misaligned and assess how the probe placement can be adjusted. Additionally, the simulation can illustrate whether any normal tissue will be affected by the ablation treatment, or whether any target tissue would be left untreated.

The ablation process is simulated with a computing device which takes into account the electrical and thermal characteristics of the tissue, the applied probe positions, geometries, the electrical and thermal characteristics of the probes, and the ablation power to be applied to each probe. This simulation provides an ablation volume prediction corresponding tissue that would be necrotized if the ablation were performed.

A visual display of the ablation volume and projected necrotized tissue is obtained, e.g., on a screen or monitor. This display can be a 2D or 3D display. If the displayed ablation volume does not encompass the tissue that the clinician would like to be necrotized, the position of, and/or energy applied to the probes is adjusted to correctly encompass the target tissue. In planning, in accordance with the present invention, this adjustment can be done using the computing system so that the probes themselves do not need to be physically moved. This saves overall treatment time. The simulation and its computed display are repeated until the ablation volume encompasses the targeted tissue. The clinician can now physically move the probes to positions resembling the simulated positions. The ablation is then performed using this probe positioning and energy.

After ablation, a scan of the tissue that has been ablated, e g., by CT, MM, PET, or an ultrasound scanner device, can be obtained and superimposed on the visual display of the ablation volume to determine the accuracy of the probe positioning and energy used for the ablation. Additional simulations and their resulting displays can be conducted simultaneously with the ablation step(s).

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Ablation Simulation Results

Using the system according to the disclosure, a simulation was setup where 3 radiofrequency multi-active probes were positioned in a liver as shown in FIGS. 3A and 3B. As illustrated, the three probes are not parallel, and their spatial position does not inform to any particular indication. A simulated radiofrequency ablation signal was then deployed.

FIGS. 4A and 4B show a simulated result after ablation using the probes as positioned in FIGS. 3A and 3B. The temporal evolution of the temperatures in the tissues was computed using a tissue damage model to determine the which tissues are necrotized, and hence the ablation volume. In the rendering above the ablated (necrotized) tissues resulting from the simulation have been rendered with a pink color, while the healthy liver tissues have been rendered with a dark brown color. Given the results of a simulation in accordance with the present invention, different views and forms of graphic presentation can be obtained, rendering the data in 3D or 2D. This rendering also can be overlaid on CT images of the liver to indicate on the images which tissues would be necrotized.

FIGS. 6 and 11 show a 2D CT image that has been generated by slicing a 3D CT volume in a plane, with the position of the necrotized tissue and the ablation volume obtained from the simulation overlaid onto the CT image of the liver. The tissues that would be necrotized according to the simulation have been highlighted in yellow color in FIG. 11. This visualization permits the clinician to evaluate in different planes, for example axial, coronal, sagittal, or any other plane, the effect of the ablation, and editor adjust the position of the probes based on the current indicated ablation volume versus the desired, or proceed to the ablation.

### Example 2

### Computing Ablation Volume Models

The ablation volume is computed using models that reflect the physics of the ablation.

The computation of the ablation volume comprises the generation of an FEM mesh that models the probes in their position in the tissues involved in the ablation (FIG. 4), and then the application of the model to the FEM mesh and its solution (FIG. 5). This process is shown in FIG. 7.

1. Ablation Volume Model in RFA

The model used for simulating the physics of RFA in the system is as follows. An ablation volume is estimated based on the position of multiple probes. This ablation volume is used to provide surgical guidance.

The RFA thermal field is described by the Pennes bioheat equation

$$ρ C \frac{\partial T}{\partial t} = ∇ \cdot k ∇ T + {Q}_{RF} + {Q}_{B} \qquad ( 0.1 )$$

where ρ is the density of the tissue, C is the specific heat capacity, T is the temperature, t is time, k is the thermal conductivity of tissues, Q_{RF} is the electrical power density deposited by the application of RF energy, Q_{B} is a term that models any biological source or sink of heat (like the heat sink effect of vessels and perfusion).

Equation (0.1) allows to compute the evolution of temperatures at the ablation site when the input parameters are specified. The parameters ρ, Care functions of the tissues, as is Q_{B}, Q_{RF} depends instead on the applied RF energy, and can be calculated using the Laplace equation:

∇·σ∇u=0 (0.2)

Where σ is the electrical conductivity of tissues and u is the electrical potential that develops in the tissues under the effect of the ablation probes. The multiple ablation probes are described with boundary condition:

$${V}_{1} = u + {zc}_{1} σ \frac{\partial u}{\partial \hat{n}} \qquad ( 0.3 )$$

where Vₗ is the voltage applied to the l-th probe, with l=1 . . . L where L is the number of probes, zcₗ is the contact impedance of the l-th probe, and n\hat{ } is the normal to the surface of the probe.

Equations (0.1) (0.2), and (0.3) are discretized spatially with the Finite Element Method and temporally with the Finite Difference Method resulting in the estimation of the temperatures T at the ablation site and over the duration of the ablation.

The computed temperatures are fed into a tissue damage model to determine which tissues will be necrotized, and hence the ablation volume. The tissue damage is modeled with the Arrhenius equation:

$$Ω = {\int }_{{t}_{s}}^{{t}_{f}} {Ae}^{\frac{- {E}_{a}}{RT ( t )}} dt \qquad ( 0.4 )$$

where Ω is the tissue damage, a function of space which takes a value of 0 where no tissues are damaged, and of 1 where tissues death is certain; A is the Arrhenius pre-exponential factor for the tissues, Eₐ is the Arrhenius activation energy for the tissue, R is the gas constant, T are the temperatures computed from (0.1), and tₛ and t_{F} are respectively the times at which the ablation is started and finished.

The scalar field Ω that results from (0.4) takes therefore values in the range [0,1]. It is common to consider the tissues with an Ω value >0.9 to be necrotized. The iso surface Ω=0.9 describes therefore the computed ablation volume resulting from the activation of the L probes in the system.

2. Ablation Volume Model in MWA

This model comprises estimating an ablation volume based on the position of multiple probes and then using this information to provide surgical guidance.

The MWA thermal field is described by the same Pennes bioheat equation used in RFA:

$$ρ C \frac{\partial T}{\partial t} = ∇ \cdot k ∇ T + {Q}_{MW} + {Q}_{B} \qquad ( 0.5 )$$

where Q_{MW} indicates now the electrical power density deposited by the application of microwave energy.

Equation (0.5) allows to compute the evolution of temperatures at the ablation site when the input parameters are specified. The parameters p, Care functions of the tissues, as is Q_{B}, Q_{MW} depends instead on the applied microwave energy, and can be calculated using the Maxwell equations:

$$∇ \cdot D = γ \qquad ( 0.6 )$$

$$∇ \cdot B = 0 \qquad ( 0.7 )$$

$$∇ \times E = - \frac{\partial B}{\partial t} \qquad ( 0.8 )$$

$$∇ \times E = \frac{\partial D}{\partial t} + J \qquad ( 0.6 )$$

where D is the electric induction vector, γ is the electric charge distribution, B is the magnetic induction, E is the electric field, H is the magnetic field, and J is the current density.

Solving equations (0.6) (0.7) (0.8) (0.9) with ad hoc boundary conditions that describe the imposed fields at the inputs of the probes and with absorbing boundary conditions and the boundaries of the computational domain allows to calculate E at the ablation site. The power density dissipated in the tissues is Q_{MW}=¹σE·E* where the symbol * indicates the complex conjugate.

The computed Q_{MW} can now be plugged into the bioheat equation (0.5) to calculate the temperatures in the tissues T. As temperatures in the tissues change, the properties of the tissues also change, and Q_{MW} ca be updated at opportune interval of times to reflect this non-linearity.

Given the computed temperatures T the Arrhenius tissue damage model (0.4) is applied to determine which tissues are necrotized and to produce and estimated ablation volume.

3. Ablation Volume Model in CRA

This model comprises estimating an ablation volume based on the position of multiple probes and then using this information to provide surgical guidance.

The CRA thermal field is described by the same Pennes bioheat equation used in RFA:

$$ρ C \frac{\partial T}{\partial t} = ∇ \cdot k ∇ T + {Q}_{CRYO} + {Q}_{B} \qquad ( 0.1 )$$

where Q_{CRYO} indicates now the heat density removed by the ablation probes; Q_{B} continues to model the biological heat sources or sinks; in the specific case of CRA vessels and perfusion became sources of heat, as tissues being ablated are at a temperature inferior to the temperature of blood. CRA simulation involves only solving (0.10) given Q_{CRYO} which is known from the type of probe being used and the settings of the ablation system.

In the context of CRA usually the temperature field T computed from (0.10) is directly used to indicate which tissues are ablated by taking iso-surfaces at a certain temperature (e.g. T=−30° C.).

3. Ablation Volume Model in IRE

In IRE, tissue damage is not cause by thermal effects as in RFA, MWA, and CRA, but instead stems from bursting of cell membranes caused by high intensity, short duration, electric pulses. The probes inserted in the tissues apply voltage pulses that diffuse in the tissues according to the Laplace equation:

∇·σ∇u=0 (0.11)

where σ is the electrical conductivity of tissues and u is the electrical potential that develops in the tissues under the effect of the ablation probes. The multiple ablation probes are described with boundary condition:

$${V}_{1} = u + {zc}_{1} σ \frac{\partial u}{\partial \hat{n}} \qquad ( 0.12 )$$

where Vₗ is the voltage applied to the l-th probe, with l=1 . . . L where L is the number of probes, zc₁ is the contact impedance of the l-th probe, and n\hat{ } is the normal to the surface of the probe.

Equation (0.11) allows computing the electric potential in the tissues u and from the electric field E=∇u. The intensity of the electric field determines whether a tissue would be subject to irreversible electroporation, the volume of the ablation can be therefore determined by the iso-surface |E|=k where k is the threshold above which electroporation occurs in the specific tissue.

4. Ablation Volume Visualization

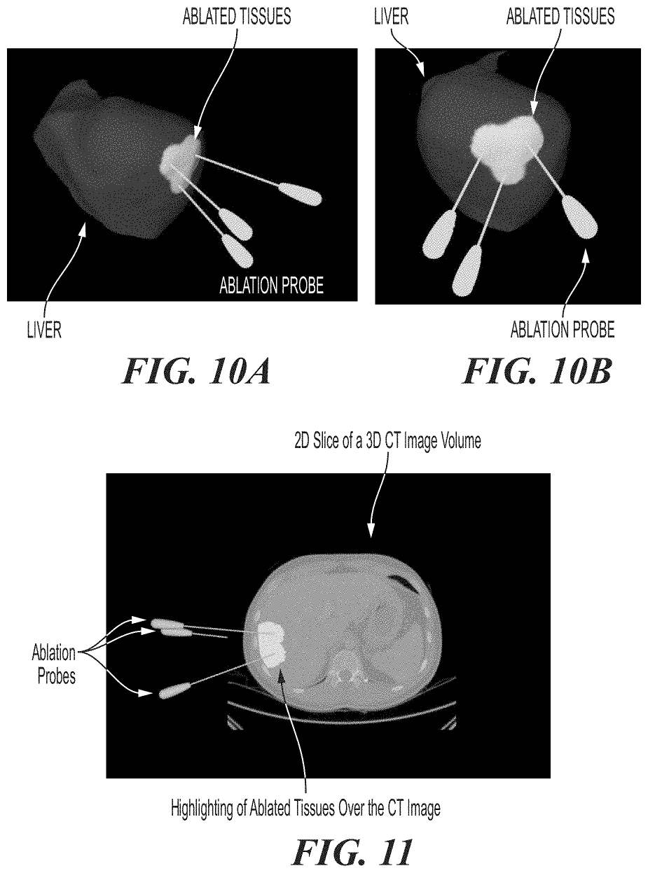

Once the ablation volume is computed, it can be visualized in a number of different ways to indicate the clinician which tissues are ablated. FIG. 8 (7) enables a clinician to explore the ablation volume inside the organ with a slice by slice representation and shows a 2-D slice of a 3D CT image which has been superimposed on the computed ablation volume. FIG. 9 (8) shows the surface of a liver to be ablated rendered in a color indicative of the tissue damage. FIG. 10 (9) is a representation of an alternate exemplary visualization example showing the organ under ablation rendered in a wireframe fashion, so that the user can see through the surface of the organ, wherein the ablation volume is represented here in pick. The user can explore which part of the organ is treated.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

A computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. A computing device may include a single computing device operating independently, or may include two or more computing device operating in concert, in parallel, sequentially or the like; two or more computing devices may be included together in a single computing device or in two or more computing devices. A computing device may interface or communicate with one or more additional devices as described below in further detail via a network interface device. Network interface device may be utilized for connecting a computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. A computing device may include but is not limited to, for example, a computing device or cluster of computing devices in a first location and a second computing device or cluster of computing devices in a second location. A computing device may include one or more computing devices dedicated to data storage, security, distribution of traffic for load balancing, and the like. A computing device may distribute one or more computing tasks as described below across a plurality of computing devices of computing device, which may operate in parallel, in series, redundantly, or in any other manner used for distribution of tasks or memory between computing devices. A computing device may be implemented using a “shared nothing” architecture in which data is cached at the worker, in an embodiment, this may enable scalability of system 100 and/or computing device.

With continued reference to FIG. 1, a computing device may be designed and/or configured to perform any method, method step, or sequence of method steps in any embodiment described in this disclosure, in any order and with any degree of repetition. For instance, a computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

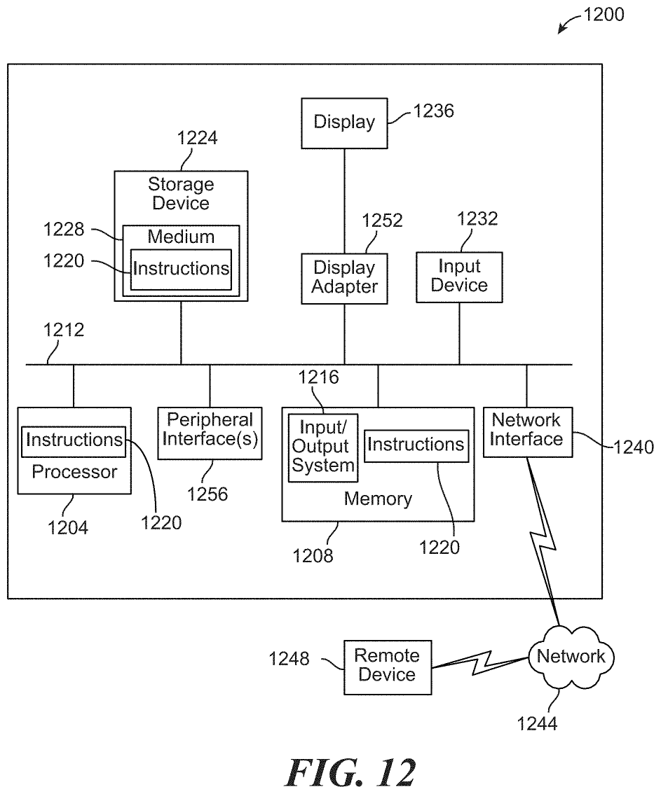

FIG. 12 shows a diagrammatic representation of one embodiment of a computing device in the exemplary form of a computer system 1200 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1200 includes a processor 1204 and a memory 1208 that communicate with each other, and with other components, via a bus 1212. Bus 1212 may include any of several types of bus structures including, but not limited to, a memory bus, a memory controller, a peripheral bus, a local bus, and any combinations thereof, using any of a variety of bus architectures.

Processor 1204 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1204 may be organized according to Von Neumann and/or Harvard architecture as a non-limiting example. Processor 1204 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1208 may include various components (e.g., machine-readable media) including, but not limited to, a random-access memory component, a read only component, and any combinations thereof. In one example, a basic input/output system 1216 (BIOS), including basic routines that help to transfer information between elements within computer system 1200, such as during start-up, may be stored in memory 1208. Memory 1208 may also include (e.g., stored on one or more machine-readable media) instructions (e.g., software) 1220 embodying any one or more of the aspects and/or methodologies of the present disclosure. In another example, memory 1208 may further include any number of program modules including, but not limited to, an operating system, one or more application programs, other program modules, program data, and any combinations thereof.

Computer system 1200 may also include a storage device 1224. Examples of a storage device (e.g., storage device 1224) include, but are not limited to, a hard disk drive, a magnetic disk drive, an optical disc drive in combination with an optical medium, a solid-state memory device, and any combinations thereof. Storage device 1224 may be connected to bus 1212 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1224 (or one or more components thereof) may be removably interfaced with computer system 1200 (e.g., via an external port connector (not shown)). Particularly, storage device 1224 and an associated machine-readable medium 1228 may provide nonvolatile and/or volatile storage of machine-readable instructions, data structures, program modules, and/or other data for computer system 1200. In one example, software 1220 may reside, completely or partially, within machine-readable medium 1228. In another example, software 1220 may reside, completely or partially, within processor 1204.

Computer system 1200 may also include an input device 1232. In one example, a user of computer system 1200 may enter commands and/or other information into computer system 1200 via input device 1232. Examples of an input device 1232 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1232 may be interfaced to bus 1212 via any of a variety of interfaces (not shown) including, but not limited to, a serial interface, a parallel interface, a game port, a USB interface, a FIREWIRE interface, a direct interface to bus 1212, and any combinations thereof. Input device 1232 may include a touch screen interface that may be a part of or separate from display 1236, discussed further below. Input device 1232 may be utilized as a user selection device for selecting one or more graphical representations in a graphical interface as described above.

A user may also input commands and/or other information to computer system 1200 via storage device 1224 (e.g., a removable disk drive, a flash drive, etc.) and/or network interface device 1240. A network interface device, such as network interface device 1240, may be utilized for connecting computer system 1200 to one or more of a variety of networks, such as network 1244, and one or more remote devices 1248 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1244, may employ a wired and/or a wireless mode of communication. In general, any network topology may be used. Information (e.g., data, software 1220, etc.) may be communicated to and/or from computer system 1200 via network interface device 1240.

Computer system 1200 may further include a video display adapter 1252 for communicating a displayable image to a display device, such as display device 1236. Examples of a display device include, but are not limited to, a liquid crystal display (LCD), a cathode ray tube (CRT), a plasma display, a light emitting diode (LED) display, and any combinations thereof. Display adapter 1252 and display device 1236 may be utilized in combination with processor 1204 to provide graphical representations of aspects of the present disclosure. In addition to a display device, computer system 1200 may include one or more other peripheral output devices including, but not limited to, an audio speaker, a printer, and any combinations thereof. Such peripheral output devices may be connected to bus 1212 via a peripheral interface 1256. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A tissue ablation system comprising:
a multi-active probe controller;
a plurality of ablation probes controlled by the controller and connected to a power source;
an imaging device;
a screen for displaying computer generated information and/or medical images; a computing system for executing programming code and algorithms; and a facility PACS network, with which the computing system interfaces.

2. The ablation system of claim 1, wherein the imaging device comprises a CT, MRI, PET, or ultrasound scanner device.

3. The ablation system of claim 1, wherein the screen is stand-alone component, or comprises a part of at least one of a Personal Computer (PC), the ablation controller, or the imaging device.

4. The ablation system of claim 1, wherein the computing system comprises a PC, an embedded system, a virtual machine, or a docker.

5. The ablation system of claim 1, wherein the computing system is directly or virtually connected to the screen.

6. The ablation system of claim 1, wherein the computing system interfaces with the controller, or to at least one ablation probe.

7. The ablation system of claim 1, wherein the computing system interfaces locally or remotely to the facility PACS network.

8. The ablation system of claim 1, wherein the computing system interfaces locally or remotely to the imaging device.

9. The ablation system of claim 1, further comprising a surgical tool tracking sub-system for tracking the intracorporeal position of the ablation probes.

10. The ablation system of claim 1, wherein the visual display is a 2D or 3D display.

11. A method of ablating a target tissue volume in a subject, comprising:
identifying the position of at least two multi-active probes in a tissue volume of a patient;
simulating at operational speed for the at least two multi-active probes the ablation process for said multi-active probes as positioned in the tissue volume with a computing device to predict the ablation tissue volume that would be necrotized if the ablation were performed;
obtaining a visual display of the ablation tissue volume, said ablation tissue volume encompassing the tissue that would be necrotized if the ablation were performed with the at least two multi-active probes positioned in the tissue volume;
adjusting the position of and/or energy provided by the at least two multi-active probes if the displayed ablation tissue volume does not encompass the target tissue to be ablated;
repeating the simulating and obtaining a visual display steps until the ablation tissue volume encompasses the targeted tissue volume, where the simulation and display steps are conducted at an operational speed allowing for an interactive update of the displayed simulation relating to the adjustment of the probe positioning and/or energy provided; and
performing the ablation.

11. The method of claim 11, wherein the ablation system is a radiofrequency ablation system, a microwave ablation system, a cryoablation system, or an irreversible electroporation ablation system.

12. The method of claim 11, wherein the positions of the at least two multi-active probes are identified with a surgical tool tracking system.

13. The method of claim 11, wherein the positions of the at least two multi-active probes are identified by acquiring an image of the tissue volume of the patient comprising the probes, and retrieving and processing the image with the computing device.

14. The method of claim 11, wherein the visual display is displayed by a screen or monitor.

15. The method of claim 11, further comprising:
obtaining an image of the target tissue volume; and
superimposing the image on the visual display of the ablation tissue volume.

16. The method of claim 11, further comprising conducting additional simulating and displaying steps simultaneously with the ablation step.

17. The method of claim 11, wherein the target tissue volume is a solid cancer or tumor.

18. The method of claim 11, wherein the target tissue volume is the atrium of the heart.

19. The method of claim 11, wherein the simulating step uses electrical and thermal characteristics of the tissue volume of the patient, the measured probe positions, the probe geometries and the electrical and thermal characteristics of the probes to predict the ablation tissue volume.

20. A method for predicting the ablation volume of an ablation procedure for ablating a target tissue volume in a subject, the method comprising:
determining relative locations of a plurality of ablation probes capable of providing ablation energy;
predicting the effect of energy collectively provided by the probes based on the determined locations to identify a simulated ablation tissue volume;
comparing the simulated ablation tissue volume with the target tissue volume;
adjusting the relative locations of the plurality of probes based on the comparison between the simulated ablation tissue volume and the target tissue volume, where the predicting step is conducted at an operational speed allowing for an interactive update of the predicted effect of energy collectively provided by the probes relative to the adjustments of the probe locations; and
predicting an associated simulated ablation tissue volume relative to the adjusted locations until the simulated ablation tissue volume encompasses the target tissue volume.
